(19) United States
(12) Patent Application Publication
(10) Pub. No.: US 2023/0380745 A1
(43) Pub. Date: Nov. 30, 2023

(54) COATED NOBLE METAL ELECTRODES WITH A COLUMN-LIKE SURFACE STRUCTURE

(71) Applicant: HERAEUS DEUTSCHLAND GMBH & CO. KG., Hanau (DE)

(72) Inventors: Andreas REISINGER, Hanau (DE); Suman NARAYAN, Hanau (DE)

(21) Appl. No.: 18/323,156

(22) Filed: May 24, 2023

(30) Foreign Application Priority Data
May 30, 2022 (DE) 102022113496.2

(51) Int. Cl. A61B 5/268

(52) U.S. Cl. CPC A61B 5/268; A61B 2562/125

(57) ABSTRACT

A medical electrode comprising a substrate, a first layer with nanocolumns applied to the substrate, and a second layer comprising an electrically conductive polymer. The first layer can be produced, for example, by means of a sputtering method. The invention also relates to production methods for these electrodes, and uses of these electrodes.

### CROSS-REFERENCE TO RELATED APPLICATIONS

This application claims priority pursuant to 35 U.S.C. 119(a) to German Application No. 102022113496.2, filed May 30, 2022, which application is incorporated herein by reference in its entirety.

### FIELD OF THE INVENTION

The present invention relates to the field of medical technology, in particular medical electrodes for use in therapeutic and diagnostic methods.

### TECHNICAL BACKGROUND

Medical electrodes can be coated with electrically conductive polymers to obtain, for example, a softer surface and/or improved electrical properties. Such coated electrodes are described, for example, in WO 2015/031265 A1.

### PREFERRED EMBODIMENTS

The object of the present invention is to provide improved coated medical electrodes and methods for the production thereof. For example, the invention enables coatings with improved mechanical stability. Furthermore, the present invention provides electrodes with improved electrical properties, such as high charge storage capacity and low impedance.

These objects are achieved by the methods and devices described herein, in particular those that are described in the claims.

Preferred embodiments of the invention are described below.

|1| A medical electrode comprising a substrate having a surface on which a first layer comprising a metal, preferably a noble metal, is arranged, wherein the first layer comprises nanocolumns, which extend in a direction R orthogonally to the surface of the substrate, and a second layer which comprises a conductive polymer and is arranged on the first layer.

|2| The medical electrode according to embodiment 1, wherein the nanocolumns are arranged in a uniform pattern.

|3| The medical electrode according to any one of the preceding embodiments, wherein the nanocolumns have a length L of 100 to 6000 nanometers along the direction R.

|4| The medical electrode according to any one of the preceding embodiments, wherein the nanocolumns have a mean distance A of 10 to 500 nanometers from one another.

|5| The medical electrode according to any one of the preceding embodiments, wherein the nanocolumns comprise a symmetrical structure, preferably a pyramidal structure.

|6| The medical electrode according to any one of the preceding embodiments, wherein the nanocolumns have a mean diameter D of 50 to 1000 nanometers.

|7| The medical electrode according to any one of the preceding embodiments, wherein the substrate and the first layer comprise the same material, or preferably consist of the same material.

|8| The medical electrode according to any one of the preceding embodiments, wherein the noble metal in the first layer comprises or consists of gold or platinum.

|9| The medical electrode according to any one of the preceding embodiments, wherein the second layer comprises PEDOT, or preferably comprises Amplicoat®.

|10| A method for producing a medical electrode, comprising the steps of:
i. providing a substrate having a surface,
ii. applying a metal to the surface by means of a coating method so that the metal on the surface forms a first layer with nanocolumns,
iii. applying a conductive polymer to the first layer.

|11| The method according to embodiment 10, wherein the coating method comprises physical vapor deposition, preferably a sputtering method, or an electrochemical coating method.

|12| The method according to embodiment 10 or 11, wherein a conductive polymer is applied to the first layer by electrochemical or chemical polymerization, and/or by means of dip coating or inkjet printing.

### DETAILED DESCRIPTION

In principle, for the embodiments described herein, the elements of which “contain” or “comprise” a particular feature (e.g., a material), a further embodiment is always considered in which the element in question consists of that feature alone, i.e., comprises no further components. The word “comprise” or “comprising” is used herein synonymously with the word “contain” or “containing.”

If an element is referred to in the singular in an embodiment, an embodiment is also considered in which a plurality of these elements are present. The use of a term for an element in the plural fundamentally also encompasses an embodiment in which only a single corresponding element is contained.

Unless otherwise indicated or clearly precluded from the context, it is possible in principle, and is herewith clearly taken into consideration, that features of different embodiments may also be present in the other embodiments described herein. It is also contemplated in principle that all features that are described herein in conjunction with a method are also applicable to the products and devices described herein, and vice versa. Only for reasons of succinct presentation are all such contemplated combinations not explicitly listed in all instances. Technical solutions which are known to be equivalent to the features described herein are also intended to be encompassed in principle by the scope of the invention.

A first aspect of the invention relates to a medical electrode comprising a substrate having a surface on which a first layer comprising a metal is arranged, wherein the first layer comprises nanocolumns, which extend in a direction R orthogonally to the surface of the substrate, and a second layer which comprises a conductive polymer and is arranged on the first layer.

The first layer comprises a noble metal. The metal preferably comprises a noble metal. Examples of noble metals are platinum, iridium, palladium, gold, ruthenium and rhodium. In one embodiment, the metal is platinum. In one embodiment, the metal is gold. The metal may also comprise an alloy, for example a platinum-iridium alloy, such as PtIr10 or PtIr20. Further examples of suitable alloys are gold-titanium alloys or nickel-titanium alloys, such as nitinol. In one embodiment, the first layer comprises a metal selected from gold, platinum and a platinum-iridium alloy.

The metal may also comprise one or more of the metals listed herein as materials for the substrate. The metal may be a biocompatible metal, as defined below. In one embodiment, the metal comprises a metal which is not copper or silver. In one embodiment, the first layer comprises a metal selected from the group consisting of gold, platinum, palladium, ruthenium, rhodium.

The medical electrode can be configured for implantation, for example into the human or animal body. In one embodiment, the medical electrode is configured for direct tissue contact. In one embodiment, the medical electrode is biocompatible. The electrode can be configured to deliver an electrical signal to the human body. The electrode can be configured to receive an electrical signal from the human body. In particular, the second layer of the medical electrode can be configured for direct contact with the body of a human or an animal. The second layer can be configured for directly receiving human or animal tissue and/or delivering thereto, in particular in the case of an implantable medical electrode. An example of a medical electrode is a ring electrode. Such ring electrodes can preferably be produced from a biocompatible metal, such as platinum, gold or platinum-iridium.

The electrode comprises a substrate that is used as a base body and to support the first layer. The electrode can comprise a flexible substrate, for example made of plastic. Examples of suitable plastics include polyimide, PTFE, PEEK, PET, LCP (liquid crystalline polymers), PU and silicones. The substrate can, for example, be a polymer film, for example a film made of PTFE or polyimide. The substrate may comprise an electrically conductive surface, for example a metal surface. The substrate can comprise a wire, for example a metal wire. The substrate can be structured and contain, for example, one or more contact elements, one or more conductor tracks, and an electrical element configured to receive and/or output an electrical signal.

The electrode can furthermore comprise an encapsulation. The encapsulation can comprise a biocompatible material, such as platinum, titanium or a medical-grade silicone. The encapsulation can comprise a feedthrough so that the active part of the electrode can be led out of the encapsulation. In one embodiment, only the active part of the structure protrudes from the encapsulation. The active part can comprise a part of the substrate and of the first layer and second layer situated thereon.

The substrate preferably comprises a smooth surface, in particular at the interface between the substrate and the first layer.

The substrate can be a partial region of a medical electrode that serves as a support layer for the first layer. The substrate can be electrically insulating or electrically conductive. In some embodiments, the substrate comprises both electrically insulating and electrically conductive elements.

The substrate can comprise a biocompatible metal. Suitable biocompatible metals are known in the art, for example Pt, Jr, Ta, Pd, Ti, Fe, Au, Mo, Nb, W, Ni, Ti, or a mixture or alloy thereof. Whether a metal or an alloy is biocompatible can be determined using the standard EN ISO 10993. Preferred biocompatible metals or alloys for use in the present invention include platinum, platinum-iridium alloys, gold and iridium, wherein iridium can preferably be used as iridium oxide.

In some embodiments, the substrate comprises or consists of the alloy MP35, PtIr10, PtIr20, 316L, 301, 304 or nitinol. The substrate can also comprise multilayer material systems. In some embodiments, the substrate consists of one or more of these materials.

MP35 is a nickel-cobalt-based curable alloy. A variant of MP35 is described in industry standard ASTM F562-13. In one embodiment, MP35 is an alloy comprising 33 to 37% Co, 19 to 21% Cr, 9 to 11% Mo, and 33 to 37% Ni.

PtIr10 is an alloy of 88 to 92% platinum and 8 to 12% iridium.

PtIr20 is an alloy of 78 to 82% platinum and 18 to 22% iridium.

316L is an acid-resistant CrNiMo austenitic steel with approx. 17% Cr; approx. 12% Ni, and at least 2.0% Mo. A variant of 316L is described in industry standard 10088-2. In one embodiment, 316L is an alloy comprising 16.5 to 18.5% Cr; 2 to 2.5% Mo, and 10 to 13% Ni.

301 is a chromium-nickel steel with high corrosion resistance. A variant of 301 is described in industry standard DIN 1.4310. In one embodiment, 301 is an alloy comprising 16 to 18% Cr and 6 to 8% Ni.

304 is an austenitic, acid-resistant 18/10 Cr—Ni steel, which is described, for example, in manufacturing standards ASTM A213, ASTM A269, ASTM A312 or ASTM A632. 304 typically contains 8-10.5% nickel, 18-20% chromium, up to 2% manganese and up to 0.08% carbon. A variant of 304 is 304L, which contains up to 12 wt. % of nickel.

Nitinol is a shape-memory nickel-titanium alloy having an ordered cubic crystal structure and a nickel content of approximately 55%, the remaining portion consisting of titanium. Nitinol has good biocompatibility and corrosion-resistance properties.

Unless otherwise indicated, all percentages given herein are to be understood as weight percent (wt. %).

Examples of biocompatible polymers include polyimide, polyethylene, polyurethane and silicone. In one embodiment, the substrate comprises polyimide, for example Kapton.

A first layer comprising a metal, for example, a noble metal, is arranged on the substrate. Examples of noble metals are platinum, iridium, palladium, gold, ruthenium and rhodium. In one embodiment, the noble metal is selected from the group consisting of platinum, iridium, palladium, gold and rhodium. The noble metal can also be a noble-metal-containing alloy, such as a platinum-iridium alloy. Examples of a platinum-iridium alloy are PtIr10 and PtIr20. In one embodiment, the first layer comprises, or consists of, platinum. In some embodiments, the first layer is free of non-noble metals or free of non-metals.

The substrate and the first layer can be formed from the same material or may comprise different materials. For example, both the substrate and the first layer can comprise platinum. In one embodiment, the first layer comprises platinum and the substrate comprises a material other than platinum. In one embodiment, the first layer comprises platinum and the substrate does not comprise any platinum.

According to the invention, the first layer has nanocolumns. These nanocolumns are arranged on a surface of the substrate. Preferably, these nanocolumns extend orthogonally to this surface of the substrate along a direction R. The structure formed by the nanocolumns can therefore be similar to that of a brush or lawn area on a microscopic level, similar to that known from self-assembled monolayers of organic molecules. However, adjacent nanocolumns are preferably spaced so far apart from one another that as a result, as rough a surface as possible is formed at a microscopic level, as described in more detail below.

For example, the nanocolumns can have a length L of 100 to 6000 nanometers along the direction R, preferably 500 to 3000 nm, more preferably 300 to 800 nm.

In some embodiments, the nanocolumns may have a mean distance A of 10 to 500 nanometers, preferably of 100 to 300 nm, from one another. This refers to the so-called “nearest neighbor” distance of the respectively adjacent nanocolumns. This distance can be determined using electron microscopy images and suitable image processing software, such as ImageJ (Rasband, W. S., ImageJ, U. S. National Institutes of Health, Bethesda, Maryland, USA, https://imagej.nih.gov/ij/, 1997-2018), for example, with the aid of the plugin “Nearest Neighbor Di stance Calculation with ImageJ,” which can be found at https://icme.hpc.msstate.edu/mediawiki/index.php/Nearest_Neighbor_Distances_Calculation_with_ImageJ.html.

For this purpose, the images should cover a range of approximately 50 to 150 iim in length. The aim here is to achieve the highest possible contrast between the substrate and the first layer. In order to evaluate the images, these gray-scale images can be converted into binary images using the “threshold” function. This means that the image pixels are in each case assigned to the background, or to the nanocolumns of the first layer, by means of a threshold value. The “threshold” method divides the image into objects and background by defining an initial threshold value. Subsequently, the mean values of the pixels at or below the threshold value and of the pixels above the threshold value are calculated. The average values of these two values are calculated, the threshold value is increased and the process is repeated until the threshold value is greater than the composite average. That is to say, threshold value=(average background+average objects)/2. The geometric parameters of the nanocolumns can then be determined from the image pixels that are assigned to the nanocolumns in this way.

This procedure can be applied accordingly for determining any geometric parameters of the nanocolumns disclosed herein, in particular to the mean distance A, the length L, and the mean diameter D of the nanocolumns, unless a different determination method is indicated herein, which would lead to a different result. In particular, cross sections of the samples can be produced by means of FIB or ion milling in order to determine the length L.

The nanocolumns can be arranged on the substrate in a regular pattern. For example, the nanocolumns can be arranged in a hexagonal pattern. The nanocolumns can also be arranged in another regular pattern, for example a pattern as is known from different two-dimensional crystal structures of different substances. The “regular patterns” according to the invention are preferably largely uniform but can have minor deviations in the symmetry, i.e., the “regular patterns” according to the invention are not necessarily “regular” to the same extent as the structure of a crystal.

The nanocolumns may comprise a substantially symmetrical structure, preferably a pyramidal structure. A pyramidal structure has a polygonal base area (i.e., a polygon as the base area), and a plurality of triangular side surfaces, which touch one another at a common point. The nanocolumns may comprise a column-shaped base structure and a pyramidal tip, e.g., similarly to the shape of an obelisk. The column-shaped base structure may have the shape of a cylinder or prism. A prism is a geometric body, which arises due to parallel displacement (extrusion) of a planar polygon along a straight line not situated in this plane in space.

The nanocolumns can have a mean diameter D of 50 to 1000 nanometers. The mean diameter D is preferably 50 to 500 nm, more preferably 100 to 700 nm.

These structural properties enable better bonding of the first layer with the second layer to be achieved. As a result, the stability of the first layer and/or the second layer on the substrate can be improved. Moreover, the aforementioned surface properties of the first layer can lead to improved electrical properties of the medical electrode according to the invention. The first layer can have a high surface roughness. For example, the first layer can have a mean surface roughness Ra of at least 500 nm. In some embodiments, the first layer has a mean surface roughness Ra of at least 500 nm, 600 nm, 700 nm, 800 nm, 900 nm, 1000 nm, 1500 nm, 2000 nm or at least 3000 nm. The surface roughness can be determined according to DIN EN ISO 25178-6:2010-06.

In particular, the first layer according to the invention can serve as a particularly advantageous support layer for the second layer. For example, conductive PEDOT-containing polymer compositions, such as Amplicoat®, have particularly good adhesion on the first layer according to the invention, in particular compared to conventional metal surfaces. The combination of first layer and second layer according to the invention can simultaneously combine special advantages of improved electrical properties and mechanical stability.

In some embodiments, the first layer comprises, on its surface, recesses that are filled with the polymer of the second layer. These recesses can be formed as intermediate spaces between the nanocolumns. In this manner, the first layer and the second layer can be interlocked.

Without wanting to commit to a particular theory, the inventors explain the improvement in stability due to the structure of the electrode according to the invention as follows:

The mechanical stability of the first layer on the substrate results at least in part from the sum of the local molecular adhesion forces at the polymer-metal interface and is thus a function of the active surface of the electrode. For an electrode surface with a certain roughness, the active surface of the electrode is a multiple (for example>5×) of the geometric area of the electrode surface. A rough electrode surface thus has a greater number of molecular anchors between the polymer film and the substrate. In one exemplary case, an electrode according to the invention has a >10-fold higher active electrode surface compared to a flat, smooth substrate, so that this electrode modification leads to a corresponding increase in the adhesion forces between the polymer and the substrate, which means improved mechanical stability.

In addition to the above-described contribution of the roughness factor to the improved adhesion forces of the film, the geometric embodiment of the noble metal structures produced according to the invention on the substrate surface additionally leads to a synergistic effect with respect to the mechanical stability of the polymer, which is based on the complementary steric filling of the nanocolumn structure of the first layer according to the invention with the polymer of the second layer, and the molecular interlocking between metal and polymer material.

This particular synergistic combination of roughness and specific geometry of the electrode produced according to the invention (steric effect) results in particularly improved polymer stability.

In addition, the fine surface structure of the first layer is protected from damage by being covered with the second layer.

The first layer can have a large specific surface area. Preferably, the first layer has a specific surface area of at least 1×10⁵ m⁻¹, preferably 1×10⁶ m⁻¹, particularly preferably at least 1×10⁷ m⁻¹, and most preferably at least 1×10⁸ m⁻¹. The specific surface area can be determined, for example, according to ISO 9277:2010.

A second layer comprising a conductive polymer is arranged on the first layer. Herein, “conductive polymer” means an electrically conductive polymer. In one aspect of the invention, it is preferred that the electrically conductive polymer comprises poly(3,4-ethylenedioxythiophene), herein also referred to as “PEDOT,” or a functionalized derivative thereof. For example, the electrically conductive polymer can be derived from 3,4-ethylenedioxythiophene (EDOT).

In some embodiments, the dielectric loss factor of the conductive polymer at 20° C. in DMSO at 100 Hz is at most 1.0, preferably at most 0.8 or 0.7.

In some embodiments, the electrical conductivity of the conductive polymer at 20° C. in DMSO is at least 50 S/cm, preferably at least 60, 70, 80, 90 or 100 S/cm.

In some embodiments, the electrical impedance of the conductive polymer at 20° C. in phosphate-buffered saline (PBS) at 1 Hz is at most 1000 ohms, preferably at most 500 ohms.

In some embodiments, the charge storage capacity of the conductive polymer at 20° C. in PBS at 1 Hz is at least 5 mC/cm2, preferably at least 10, 20 or 30 mC/cm².

In one aspect of the invention, it is preferred that the electrically conductive polymer is derived from a functionalized derivative of EDOT, which is selected from the group consisting of hydroxymethyl EDOT, EDOT vinyl, EDOT ether allyl, EDOT COOH, EDOT MeOH, EDOT silane, EDOT vinyl, EDOT acrylate, EDOT sulfonate, EDOT amine, EDOT amide and combinations thereof. For example, the functionalized derivative of 3,4-ethylenedioxythiophene (EDOT) can be selected from the group consisting of hydroxymethyl EDOT, EDOT vinyl, EDOT ether allyl, EDOT acrylate and combinations thereof.

“Functionalized derivative” or “derivative” herein refers to variants of a chemical compound in which a hydrogen atom is replaced by any other substituent.

In one aspect of the invention, it is preferred that the electrically conductive polymer contains an anionic photoreactive crosslinking agent. In this aspect, it is preferred that the crosslinking agent comprises at least two photoreactive groups. In a further aspect of the invention, it is preferred that the anionic photoreactive crosslinking agents comprise a compound of Formula I:

Xi˜Y˜X2
wherein Y is a functional group containing at least one acidic group or salt of an acidic group; and Xi and X2 are each independently a functional group containing a latent photoreactive group. Examples of a photoreactive group are an aryl ketone or a quinone. In a further aspect of the invention, it is preferred that spacers are part of Xi or X2, preferably together with the latent photoreactive group.

In one aspect of the invention, it is preferred that, in the compound of Formula I Y is a functional group comprising at least one acidic group or salt thereof. Examples of acidic groups include sulfonic acids, carboxylic acids, phosphonic acids and the like. Examples of salts of such groups are sulfonate, carboxylate and phosphate salts. As an example, the crosslinking agent can contain a sulfonic acid or sulfonate group. In a further aspect of the invention, it is preferred that such a photoreactive crosslinking agent is anionic. Examples of counterions are alkali and alkaline earth metals, ammonium, protonated amines and the like.

In one aspect of the invention, it is preferred that the electrically conductive polymer comprises an anionic photoreactive hydrophilic polymer. In this aspect, it is preferred that the hydrophilic polymer is anionic. Examples of anionic hydrophilic polymers include homopolymers, copolymers, terpolymers and the like. In a further aspect of the invention, it is preferred that the anionic hydrophilic polymer is derivatized with photoreactive groups if the electrically conductive polymer comprises at least one anionic hydrophilic polymer.

In a further aspect of the invention, it is preferred that the anionic hydrophilic polymer comprises polymers containing polyacrylamide and photoreactive groups (“photo-PA”). In a further aspect of the invention, it is preferred that the anionic hydrophilic polymer comprises polyacrylamide and sulfonate groups. For example, the anionic hydrophilic polymer comprises acrylamido-2-methylpropane sulfonate (AMPS) groups and polyethylene glycol segments.

The terms “latent photoreactive group” and “photoreactive group” are used synonymously and refer to a chemical entity that is sufficiently stable to remain in an inactive state (i.e., ground state) under normal storage conditions but can undergo a transformation from the inactive state to an activated state if exposed to a suitable energy source. Unless otherwise specified, the reference to photoreactive groups preferably also includes the reaction products of the photoreactive groups.

In one aspect of the invention, it is preferred that the photoreactive groups are selected to be responsive to different parts of the actinic radiation. For example, groups can be selected to be photoactivated with either ultraviolet or visible radiation. Examples of photoreactive groups include azides, diazos, diazirines, ketones and quinones. In a further aspect of the invention, it is preferred that the photoreactive group comprises an aryl ketone, such as acetophenone, benzophenone, anthrone, and anthrone-like heterocycles (i.e., heterocyclic analogs of anthrone, such as those with N, O or S at the 10-position), or substituted (e.g., ring-substituted) derivatives thereof. Examples of aryl ketones include heterocyclic derivatives of anthrone, including acridone, xanthone, and thioxanthone, as well as their ring-substituted derivatives. Other suitable photoreactive groups are quinones, such as anthraquinone.

Electrically conductive polymers are known to those skilled in the art and are commercially available under the brand names Orgacon®, available from Agfa-Gevaert N.V. (Belgium), or Amplicoat®, available from Heraeus Deutschland GmbH & Co. KG (Germany). Further examples of electrically conductive polymers, along with methods for applying them to substrates, are described in WO 2015/031265 A1, which is hereby incorporated by reference in its entirety.

In a further aspect of the invention, it is preferred that the second layer comprises a biocompatible polymer. In a further aspect of the invention, it is also preferred that the second layer is hydrophilic. A “hydrophilic” material is defined as one that has a water contact angle of less than 90°.

In a further aspect of the invention, it is preferred that the second layer has a water contact angle that is in the range of 10° to 30°, preferably in the range of 15° to 25° and particularly preferably in the range of 19° to 22°. In a further aspect of the invention, it is preferred that the second layer has a surface energy that is in the range of 35 mN/m to 55 mN/m, more preferably in the range of 40 mN/m to 50 mN/m and further preferably in the range of 42 mN/m to 46 mN/m.

Other suitable conductive polymers are described, for example, in WO 2015/031265 A1, which is hereby incorporated by reference.

In some embodiments, the thickness of the first layer is less than 3 μm, for example less than 2 μm or less than 1 μm. In some embodiments, the thickness of the first layer is less than 900, 800, 700, 600 or less than 500 nm.

In some embodiments, the thickness of the second layer is at least 300 nm, more preferably at least 400, 500, 600, 700, 800, 900 or 1000 nm. In some embodiments, the thickness of the second layer is approximately 500 to approximately 1000 nm or approximately 800 to 2000 nm.

The thickness of the respective layers (for example, the first layer and the second layer) can be determined by evaluating cross-sectional electron microscopy images, wherein the average distance between the profile lines along the opposing interfaces of a layer to be determined is calculated using suitable image processing software. More details on the determination of the layer thickness by means of electron microscopy are described in Giurlani et al, Coatings 2020, 10, 1211; doi:10.3390/coatings10121211.

In some embodiments, the mean distance A of adjacent nanocolumns is at most as large as twice the mean diameter D of the nanocolumns. This means that the nanocolumns are packed very tightly.

In some embodiments, the second layer intercalates with the first layer. This means that the material of the second layer fills substantially all the interstices and cavities of the first layer so that there is an interpenetrating, closed composite of two different materials.

In order to further improve the stability of the electrode, the edge of the second layer can be protected by applying a third layer to the edge of the second layer. As used herein, “edge” of the second layer means the lateral end or lateral edge of the second layer, not the entire remaining surface of the second layer facing outward in the direction away from the substrate.

For example, the edge of the second layer can be covered with a polymer layer. Preferably, the second layer should not be completely covered, but as large a part of the second layer as possible should remain freely accessible to the outside, so that the third layer influences the electrical properties of the electrode as little as possible. With the aid of the above-described arrangement of a third layer, the adhesion of the first and/or second layer to the substrate can be improved even further.

A further aspect of the invention relates to a method for producing a medical electrode. The method may comprise the steps of:
i. providing a substrate having a surface,
ii. applying a metal to the surface by means of a coating method so that the metal on the surface forms a first layer with nanocolumns,
iii. applying a conductive polymer to the first layer.

The first layer can be applied by various coating methods. Preferably, the first layer can be produced by a sputter coating method. As a result, on the one hand, the nanocolumns described herein can be produced and, at the same time, very thin layer thicknesses with correspondingly low material consumption can be achieved. This is very advantageous, in particular in the case of expensive noble metals, such as platinum. Moreover, sputtering methods can be carried out on a multiplicity of different substrate materials. By means of sputtering methods, different surface structures can be produced by selecting suitable process conditions.

The selection of suitable process conditions and the relationship with the structures formed is described in numerous scientific publications, for example THORNTON, Journal of Vacuum Science and Technology 11, 666 (1974); THORNTON, Journal of Vacuum Science and Technology 12, 830 (1975); THORNTON, Ann. Rev. Mater. Sci. 1977, 7:239-60.; and THORNTON, Journal of Vacuum Science & Technology A 4, 3059 (1986).

In principle, similar layers can also be applied using other coating methods. For example, metals, in particular also noble metals, such as platinum, can be applied with the aid of wet-chemical redox reactions or electrochemical reactions, such as BOEHLER et al., Biomaterials 67 (2015).

A sputter coating method is a physical vapor deposition technique in which a glow discharge is produced with an electrode attached to a film material (target) as cathode in a vacuum container into which an inert gas, such as argon is introduced, so that ions are generated therein. These ions collide with the cathode with an energy of several hundred electron volts, which corresponds to the discharge voltage, to form a film on a substrate by the deposition of particles, e.g., metal atoms, which are released as a reaction of the collision. The method is also referred to as cathode sputtering.

Furthermore, it is contemplated that other physical vapor deposition methods can also be used, such as thermal vapor deposition, electron beam evaporation, pulsed laser deposition, arc evaporation (Arc-PVD), molecular beam epitaxy, ion plating, ionized cluster beam deposition, or ion beam assisted deposition (IBAD).

The first layer may consist substantially of the nanocolumns described herein, or it may be a mixed layer. Such a mixed layer may contain various structures that may be formed from the same material. For example, firstly a closed platinum layer can be applied to the substrate, and nanocolumns made of platinum can thereafter be applied to this closed platinum layer. Using a sputtering method, such a mixed layer can be applied to the substrate in a common working step, wherein the process parameters are adapted accordingly during coating so that the corresponding structures are formed.

The application of a conductive polymer to the first layer can take place, for example, by means of dip coating or inkjet printing. The curing of the polymer can take place by means of chemical or electrochemical polymerization. The PEDOT and similar polymers can preferably be cured electrochemically.

A further aspect of the invention relates to the use of any of the methods described herein for producing a medical electrode.

A further aspect of the invention relates to a medical electrode that can be produced by the methods described herein.

In a further aspect, an electrical medical device comprising an electrode according to any one of the preceding aspects and embodiments thereof is provided.

The electrical medical device can, for example, be a lead for use with a pulse generator, cardiac pacemaker, cardiac resynchronization device, mapping catheter, sensor or stimulator. Leads are electrical wires which can be used, for example, in medical applications, such as cardiac stimulation, neuromodulation, deep-brain stimulation, spinal-cord stimulation, or gastric stimulation. In one embodiment, the lead is configured and/or intended to be connected to a generator of an active implantable device. A lead can also be used in a medical device to receive an electrical signal from the body of a living being. A stimulator is a medical device that can achieve a physiological effect by sending an electrical signal to the body of a living being. For example, a neurostimulator can, by delivering an electrical signal to a nerve cell, produce an electrical signal in the nerve cell (e.g., an action potential).

A further embodiment relates to a microelectrode array containing a plurality of electrodes according to the invention.

A further aspect relates to a diagnostic method in or on the body of a living being, comprising the recording of an electrical signal by means of the electrode described herein.

A further aspect relates to the use of the electrode described herein in a diagnostic method in or on the body of a living being, comprising the recording of an electrical signal by means of the electrode.

A further aspect relates to a therapeutic method in or on the body of a living being, comprising the delivery of an electrical signal by means of the electrode described herein.

A further aspect relates to the use of the electrode described herein in a therapeutic method in or on the body of a living being, comprising the delivery of an electrical signal by means of the electrode.

The therapeutic method can comprise the delivery of an electrical signal to nerve cells or muscle cells in the region of an organ, for example, the heart, muscle, stomach or brain.

The diagnostic method can comprise the recording of an electrical signal from nerve cells or muscle cells in the region of an organ, for example, heart, muscle or brain.

### EXAMPLES

The invention is further illustrated below using examples, which are, however, not to be understood as limiting. It will be apparent to a person skilled in the art that other equivalent means may be similarly used in place of the features described here.

### Example 1: Production of a Nanocolumn-Containing Layer on a Medical Ring Electrode

With the aid of a magnetron sputter coating method, medical ring electrodes made of platinum-iridium were provided with a first nanocolumn-containing layer made of platinum. Thereafter, these samples were coated with a biocompatible conductive polymer according to the manufacturer's instructions (Amplicoat™, available from Heraeus, Hanau, Germany).

Identical ring electrodes, on which no nanocolumn-containing layer was applied, served for comparison. Some of these comparative samples were treated with sodium bicarbonate by mechanical grinding or shot peening to increase surface roughness.

The ring electrodes had an outer surface of 31.0 mm2, a diameter of approximately 1.2 mm, and a length of approximately 4.2 mm.

The layer thickness of the applied Amplicoat™ layer is related to the charge storage capacity (CSC) of the samples. The higher the CSC value, the thicker the coating. Samples coated with an approximately 500 nm thick layer of Amplicoat™ had a charge storage capacity of about 12,000 mC/cm².

After coating, all samples were subjected to a visual inspection and electrochemical characterization was carried out using potentiostatic electrochemical impedance spectroscopy (PEIS).

In order to in particular examine the stability of the second layer (Amplicoat), a wipe test was carried out in which a certain weight was applied to the coating and was wiped back and forth 10 times in each direction. The weight was increased in steps of 20 g each time, starting with 43 g up to a maximum value of 203 g. Each sample was thus smeared a total of 180 times during each test. The contact surface was 2 mm².

After each wipe test, the samples were again subjected to a visual inspection to check delamination of the second layer (Amplicoat).

In the wipe test, the samples without the nanocolumn-containing layer showed distinct delamination even at the lowest weight of 43 g, i.e., a partial detachment of the second layer was observed. Furthermore, the mechanically roughened samples without a nanocolumn-containing layer were tested again with a lower weight of approximately 10 to 20 g (weight of the apparatus). Here too, distinct delamination could already be observed.

The samples that were roughened by means of shot peening and had no nanocolumn-containing layer were incubated for seven days at 55° C. in PBS (phosphate-buffered saline). In this case, the second layer detached without mechanical impact.

In contrast, the samples with a nanocolumn-containing layer did not demonstrate any visual delamination, even at the highest weight tested of 203 g, but remained stable.

By measuring the charge storage capacity before and after the wipe test, it was confirmed that the charge storage capacity did not change significantly due to the wipe test, i.e., the Amplicoat layer was not substantially impaired.

It follows from this that the nanocolumn-containing layers according to the invention improve the adhesion of electrically conductive polymers, such as Amplicoat, to a substantially greater extent than an increase in the surface roughness with conventional means.

TABLE 1
Measurement results from Example 1.

| | | Charge Storage |
|---|---|---|
| | Impedance | Capacity CSC |
| Sample | [ohms] | [mC/cm2] |
| Pt ring electrode | 17337.9 | −2.323 |
| Pt ring electrode | 639.835 | −7.816 |
| with nanocolumn-containing | | |
| layer | | |
| Pt ring electrode + | 143.496 | −11.522 |
| Amplicoat ™ | | |
| Pt ring electrode | 139.796 | −12.349 |
| with nanocolumn-containing | | |
| layer + Amplicoat ™ | | |

### FIGURES

FIG. 1 shows, by way of example, a schematic section of a medical electrode according to the invention. The electrode comprises a substrate 101 surface 102. A first layer 103 is arranged on the surface 102. The first layer 103 comprises nanocolumns 104. A second layer 105 comprising a conductive polymer is arranged on the first layer 103. The conductive polymer surrounds the nanocolumns. The nanocolumns are thereby embedded in the conductive polymer so that the first layer 103 is interlocked with the second layer 105. In this way, a particularly good adhesion of the second layer 105 can be achieved.

FIG. 2 shows an enlarged detail of FIG. 1. The nanocolumns 104 extend along a direction R orthogonally to the surface 102. The nanocolumns 104 have a diameter D in a direction parallel to the surface 102. Along the direction R, the nanocolumns have a length L which corresponds to the distance from the surface 102 to the tip of a nanocolumn 104. The respectively adjacent nanocolumns are each arranged at a mean distance A from one another, wherein the distance A is defined as the distance between the respective center points of the adjacent nanocolumns 104.

FIG. 3 shows a perspective schematic view of a nanocolumn 104. The nanocolumn 104 comprises a pyramid-shaped tip and a column-shaped base structure having the shape of a prism or cylinder.

FIG. 4 shows an electron microscopy image of a first layer according to the invention in plan view. The pyramid-shaped tips of the individual nanocolumns can be clearly seen. In this example, the nanocolumns have a very dense surface packing, i.e., the order of magnitude of the mean distance A is in a similar range to the average diameter D of the nanocolumns.

FIG. 5 shows an electron microscopy image of a first layer according to the invention in cross section. Visually, the second layer 102 in this figure is distinctly different from the first layer 101. The thickness of the second layer 102 is between 1 and 10 μm.

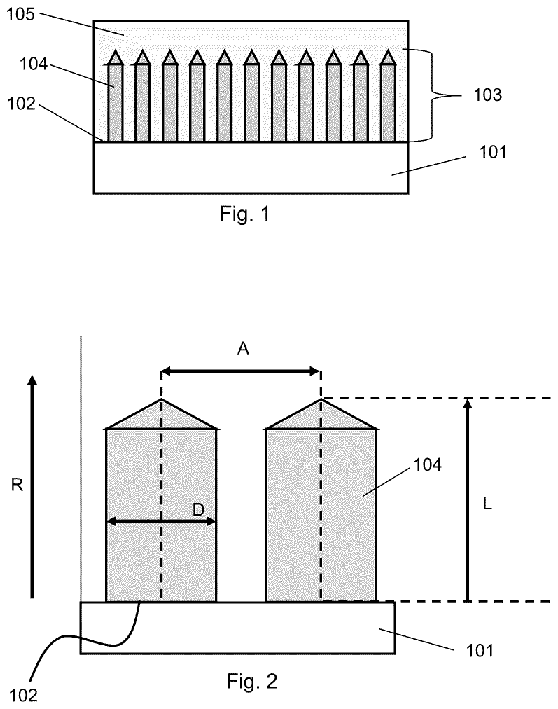

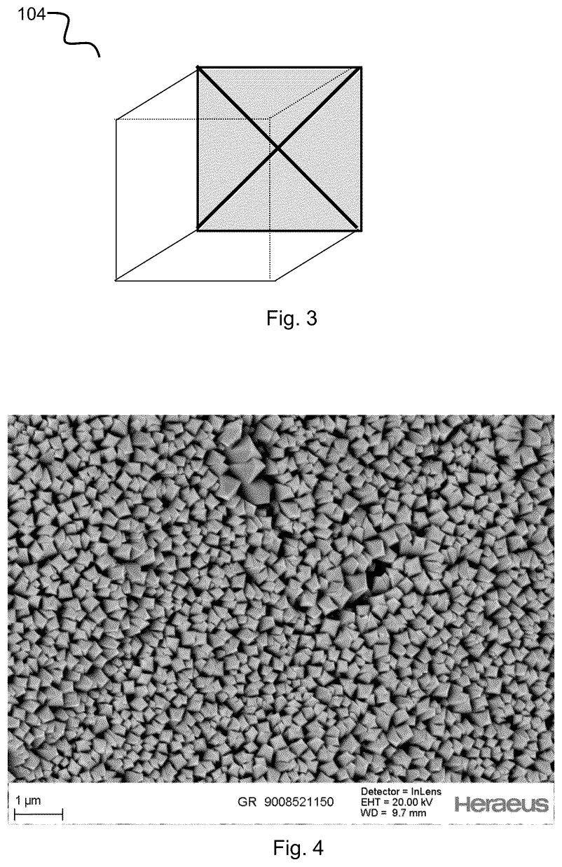

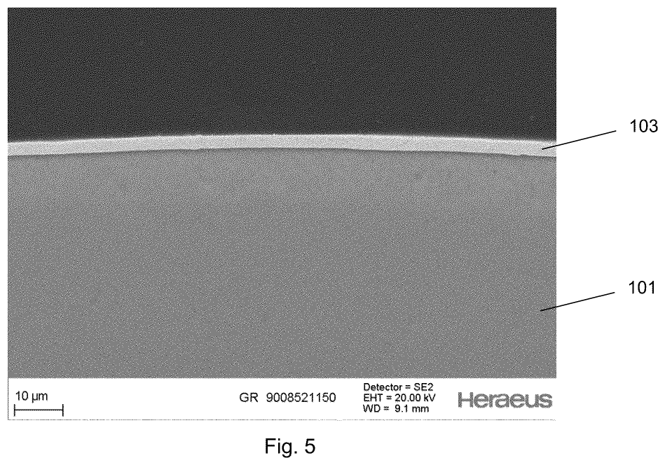

What is claimed is:

1. A medical electrode comprising a substrate having a surface on which a first layer comprising a metal, preferably a noble metal, is arranged, wherein the first layer comprises nanocolumns extending in a direction R orthogonally to the surface of the substrate, and a second layer which comprises a conductive polymer and is arranged on the first layer.

2. The medical electrode according to claim 1, wherein the nanocolumns are arranged in a uniform pattern.

3. The medical electrode according to claim 1, wherein the nanocolumns have a length L of 100 to 6000 nanometers along the direction R.

4. The medical electrode according to claim 1, wherein the nanocolumns have a mean distance A of 10 to 500 nanometers from one another.

5. The medical electrode according to claim 1, wherein the nanocolumns comprise a symmetrical structure, preferably a pyramidal structure.

6. The medical electrode according to claim 1, wherein the nanocolumns have a mean diameter D of 50 to 1000 nanometers.

7. The medical electrode according to claim 1, wherein the substrate and the first layer comprise the same material, or preferably consist of the same material.

8. The medical electrode according to claim 1, wherein the noble metal in the first layer comprises or consists of gold or platinum.

9. The medical electrode according to claim 1, wherein the second layer comprises PEDOT, or preferably comprises Amplicoat®.

10. A method for producing a medical electrode, comprising the steps of:
(i) providing a substrate having a surface,
(ii) applying a metal to the surface by means of a coating method so that the metal on the surface forms a first layer with nanocolumns,
(iii) applying a conductive polymer to the first layer.

11. The method according to claim 10, wherein the coating method comprises physical vapor deposition, preferably a sputtering method, or an electrochemical coating method.

12. The method according to claim 10, wherein a conductive polymer is applied to the first layer by electrochemical or chemical polymerization, or by means of dip coating or inkjet printing.
